(19) United States
(12) Patent Application Publication
(10) Pub. No.: US 2018/0236690 A1
(43) Pub. Date: Aug. 23, 2018

(54) SYSTEM AND METHOD FOR INSPECTION OF CONCRETE BLOCKS

(71) Applicant: NEOCULUS TECHNOLOGIE INC., Quebec (CA)

(72) Inventors: Pierre-Luc SAINDON, Quebec (CA); Christian SAINDON, Quebec (CA); Marco GILBERT, Quebec (CA); Patrick NOLET, Quebec (CA)

(21) Appl. No.: 15/438,316

(22) Filed: Feb. 21, 2017

(51) Int. Cl. B28B 17/00; E04C 1/00; G01N 9/02; G01N 33/38

(52) U.S. Cl. CPC B28B 17/0072; E04C 1/00; G01N 2009/024; G01N 33/383; G01N 9/02

(57) ABSTRACT

A system for performing the inspection of a concrete block includes a conveyor, a weighing module and a scanning module. The conveyor extends along a conveying axis and conveys a support plate with the concrete block supported thereon. The weighing module weighs the support plate with the concrete block supported thereon. The scanning module includes a scanner scanning the upper surface of the concrete block and the upper surface of the support plate. The scanner is displaceable along a scanning axis and has a scanning range covering an entirety of an upper surface of the concrete block supported on the support plate and a section of the upper surface of the support plate extending from at least two consecutive lower edges of the concrete block supported on the support plate. A method performs the inspection of a concrete block.

### TECHNICAL FIELD OF THE INVENTION

The present invention relates to the field of industrial inspection. More particularly, it relates to a system for performing inspection of concrete blocks and to a method for performing the inspection of concrete blocks using the same.

### BACKGROUND

Concrete blocks are commonly produced by filling a hollow mold with a concrete mixture and compressing the concrete mixture to produce molded uncured blocks having the required block size, shape and density. Such a process is commonly performed by operators producing the concrete mixture and performing the compression thereof. The produced molded uncured blocks can subsequently be cured to generate the finished concrete blocks currently available in the market.

In such a process, human error and/or defective parameters or operation of the material used for the production of the molded uncured blocks can lead to default (or non-conformity) in the aesthetic, dimensional and/or structural attributes of the produced molded uncured blocks (and consequently in the manufactured finished concrete blocks). Hence, to ensure a constant quality of the manufactured concrete blocks, it is desirable to repeatedly perform quality testing of the molded uncured blocks and/or finished concrete blocks.

It is common to carry these tests through repetitive manual inspection of dimensional attributes of a sample of the molded uncured blocks and/or finished concrete blocks by an inspector, for example through manual measuring of predetermined measure sections (or measure points) thereof. Such manual inspections can also include the weighing of the blocks to calculate the density thereof, using the measures of the predetermined sections as data for the height of the blocks. Such manual inspection suffers from several drawbacks. For example, and without being limitative, the inspection of only a sample of blocks can lead to untested blocks having defaults not being flagged as non-conform. Moreover, it can lead to fluctuations in the quality of the tests depending on the skills of the persons performing the tests. Furthermore, the delays between the inspection of two samples can lead to the production of a plurality of blocks before the detection of a defect in the characteristics of the material used for the production of the molded uncured blocks or in the operation of the machinery, thereby increasing the number of unusable blocks being produced and contributing to production loss.

Some of the above-mentioned drawbacks can be alleviated by using known automated measuring system, for example using non-contact measuring technology such as laser measuring sensors or the like. Known automated measuring system however also tend to suffer from several drawbacks. For example and without being limitative, such systems can often only perform a scan of a section (e.g. a linear section) of a block conveyed under the measuring sensors, thereby limiting the detection of defaults to the sections where the measure is performed. Moreover, such systems typically perform a calculation of the height of a block by using an average of the measured heights of the blocks along the scanned section, which limits the accuracy of the measure, especially for highly porous blocks. Known system can also limit the production speed due to the accumulation of measuring delays and can be prone to measure inaccuracy resulting from vibration of the system during the measure periods. Known system also cannot perform the inspection of blocks having a textured upper surface.

In view of the above, there is a need for an improved system and method for the inspection of concrete blocks which would be able to overcome or at least minimize some of the above-discussed prior art concerns.

### BRIEF SUMMARY OF THE INVENTION

In accordance with a first general aspect, there is provided a system for performing the inspection of a concrete block. The system comprises a conveyor, a weighing module and a scanning module. The conveyor extends along a conveying axis and conveys a support plate having an upper surface and the concrete block supported thereon. The concrete block has an upper surface, a lower surface, side walls extending between the upper surface and the lower surface and lower edges defined at a junction of the side walls and the upper surface of the support plate. The weighing module weighs the support plate with the concrete block supported thereon. The scanning module includes a scanner scanning the upper surface of the concrete block and the upper surface of the support plate. The scanner is displaceable along a scanning axis and has a scanning range covering an entirety of the upper surface of the concrete block supported on the support plate and a section of the upper surface of the support plate extending from at least two consecutive ones of the lower edges of the concrete block supported on the support plate.

In an embodiment, the weighing module engages the support plate being conveyed on the conveyor from below and the scanner is positioned above the support plate being conveyed on the conveyor and the concrete block supported thereon.

In an embodiment, the weighing module includes a lifting mechanism configured to lift the support plate away from the conveyor during a weighing time period where the weighing module performs the weighing of the support plate with the concrete block supported thereon.

In an embodiment, the weighing module includes a vibration isolation assembly substantially isolating the support plate engaged by the weighing module from vibrations, during the weighing thereof.

In an embodiment, the weighing module and the scanning module are substantially aligned along the conveying axis to perform the scanning of the upper surface of the concrete block and the support plate during the weighing time period.

In an embodiment, the scanning module includes a support base with the scanner mounted thereto and supported thereon. The support base comprises a vibration isolation assembly substantially isolating the scanner from vibrations.

In an embodiment, the support base includes a guide rail extending along the scanning axis and the scanner is secured to a slider slidably engaged with the guide rail. The scanning module further comprises a linear actuator connected between the scanner and the base and configured to move the scanner along the scanning axis.

In an embodiment, the weighing module generates weight data and the scanning module generates surface scan data. The system further comprises a computing unit in data communication with the weighing module and the scanning module. The computing unit receives the weight data from the weighing module and the surface scan data from the scanning module, processes the weight data and the surface scan data according to a set of instructions and generates inspection data indicative of a conformity or non-conformity of the concrete block.

In an embodiment, the concrete block supported on the support plate includes a predetermined measure section of the upper surface thereof. The computing unit is configured to process the surface scan data according to the set of instructions to define a set of topmost surface points of the predetermined measure section and to generate a virtually defined substantially planar topmost surface aligned on top of the set of topmost surface points.

In an embodiment, the computing unit is configured to process the weight data and the surface scan data according to the set of instructions to determine a volume and a weight of the concrete block. The volume and the weight of the concrete block are used to determine a density of the concrete block.

In an embodiment, to determine the volume of the concrete block, the computing unit is configured to process the surface scan data according to the set of instructions to define a plane of the lower surface of the concrete block from the surface scan data relative to the section of the upper surface of the support plate extending from the at least two consecutive ones of the lower edges of the concrete block supported on the support plate, generate XYZ coordinates for surface points defining the lower surface of the concrete block extending along the defined plane thereof and calculate a sum of height differences between each surface point defining the upper surface of the concrete block and a corresponding one of the surface points defining the lower surface of the concrete block.

In accordance with another general aspect, there is also provided a method for performing the inspection of a concrete block. The method comprises: conveying a support plate having an upper surface and a concrete block supported thereon along a conveying axis, the concrete block having an upper surface, a lower surface, side walls extending between the upper surface and the lower surface and lower edges defined at a junction of the side walls and the upper surface of the support plate; weighing the support plate with the concrete block supported thereon; and displacing a scanner along a scanning axis to scan the upper surface of the concrete block and the upper surface of the support plate as the concrete block and the support plate remain static, the scan covering an entirety of the upper surface of the concrete block supported on the support plate and a section of the upper surface of the support plate extending from at least two consecutive ones of the lower edges of the concrete block supported on the support plate.

In an embodiment, the step of weighing the support plate with the concrete block supported thereon includes engaging the support plate from below.

In an embodiment, the support plate is conveyed on a conveyor and the step of weighing the support plate with the concrete block supported thereon includes lifting the support plate away from the conveyor during a weighing time period.

In an embodiment, the step of weighing the support plate with the concrete block supported thereon includes substantially isolating the support plate from vibrations during the weighing time period.

In an embodiment, the step of weighing the support plate with the concrete block supported thereon and the step of displacing a scanner along the scanning axis to scan the upper surface of the concrete block and the upper surface of the support plate are performed simultaneously.

In an embodiment, the step of weighing the support plate with the concrete block supported thereon further comprises generating weight data and the step of displacing the scanner along the scanning axis to scan the upper surface of the concrete block and the upper surface of the support plate further comprises generating scan surface data. The method further comprises processing the weight data and the surface scan data according to a set of instructions and generating inspection data indicative of a conformity or a non-conformity of the concrete block with specifications thereof.

In an embodiment, the concrete block supported on the support plate includes a predetermined measure section of the upper surface thereof and the step of processing the weight data and the surface scan data according to a set of instructions comprises defining a set of topmost surface points of the predetermined measure section and generating a virtually defined substantially planar topmost surface aligned on top of the set of topmost surface points.

In an embodiment, the step of processing the weight data and the surface scan data according to a set of instructions comprises determining a volume and a weight of the concrete block, the volume and the weight of the concrete block being used to determine a density of the concrete block.

In an embodiment, the step of determining a volume of the concrete block includes determining a plane of the lower surface of the concrete block using the scan surface data relative to the section of the upper surface of the support plate extending from the at least two consecutive ones of the lower edges of the concrete block supported on the support plate; generating XYZ coordinates for surface points defining the lower surface of the concrete block extending along the plane thereof; and calculating a sum of the height differences between each surface point defining the upper surface of the concrete block and a corresponding one of the surface points defining the lower surface of the concrete block.

### BRIEF DESCRIPTION OF THE DRAWINGS

Other objects, advantages and features will become more apparent upon reading the following non-restrictive description of embodiments thereof, given for the purpose of exemplification only, with reference to the accompanying drawings in which:

FIGS. 1A to 1C are perspective views of the system for performing inspection of concrete blocks, in accordance with an embodiment, wherein, in FIG. 1A the system is shown with all components, in FIG. 1B the system is shown with the conveyor removed and in FIG. 1C the system is shown with the conveyor and a protective cover of the scanning module removed.

FIGS. 2A and 2B are respectively a side elevation view and a perspective view of the weighing module of the system for performing inspection of concrete blocks of FIG. 1A, with the protective covers removed.

FIGS. 3A to 3C are respectively an isometric view, a front elevation view and a side elevation view of the scanning module of the system for performing inspection of concrete blocks of FIG. 1A, with the protective covers removed.

FIGS. 4A and 4B are respectively a side view and a top view of a graphical representation of the scanning range of the scanner of the scanning module of the system for performing inspection of concrete blocks of FIG. 1A.

FIG. 5 is an image showing a topographic view of inspected concrete blocks, using the system for performing inspection of concrete blocks of FIG. 1A.

FIGS. 6 and 6A are respectively a cross section view of a concrete block inspected using the system for performing inspection of concrete blocks of FIG. 1A and an enlarged view of a predetermined measure section of the concrete block.

FIG. 7 is a cross-sectional view of concrete blocks inspected using the system for performing inspection of concrete blocks of FIG. 1A and supported on a support plate.

### DETAILED DESCRIPTION

In the following description, the same numerical references refer to similar elements. The embodiments, geometrical configurations, materials mentioned and/or dimensions shown in the figures or described in the present description are embodiments only, given solely for exemplification purposes.

Although the embodiments of the system for performing inspection of concrete blocks and corresponding parts thereof consist of certain geometrical configurations as explained and illustrated herein, not all of these components and geometries are essential and thus should not be taken in their restrictive sense. It is to be understood, as also apparent to a person skilled in the art, that other suitable components and cooperation thereinbetween, as well as other suitable geometrical configurations, may be used for the system for performing inspection of concrete blocks, as will be briefly explained herein and as can be easily inferred herefrom by a person skilled in the art. Moreover, it will be appreciated that positional descriptions such as “above”, “below”, “left”, “right” and the like should, unless otherwise indicated, be taken in the context of the figures and should not be considered limiting.

Moreover, although the embodiments as illustrated in the accompanying drawings comprises steps of a method for performing inspection of concrete blocks, not all of these steps are essential and thus should not be taken in their restrictive sense. It is to be understood, as also apparent to a person skilled in the art, that other suitable steps or sequence of operation may be used for the method, as will be briefly explained herein and as can be easily inferred herefrom, by a person skilled in the art, without departing from the scope of the invention.

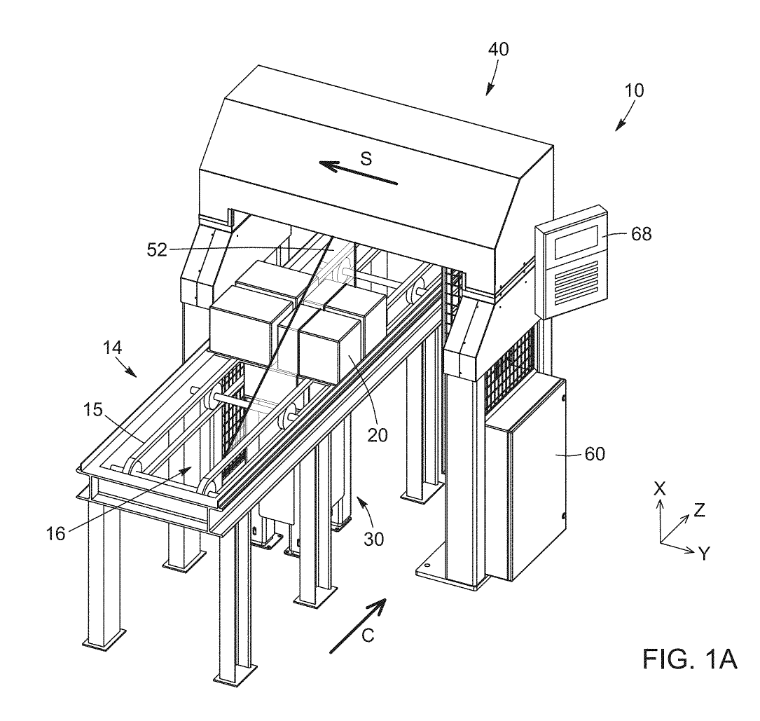

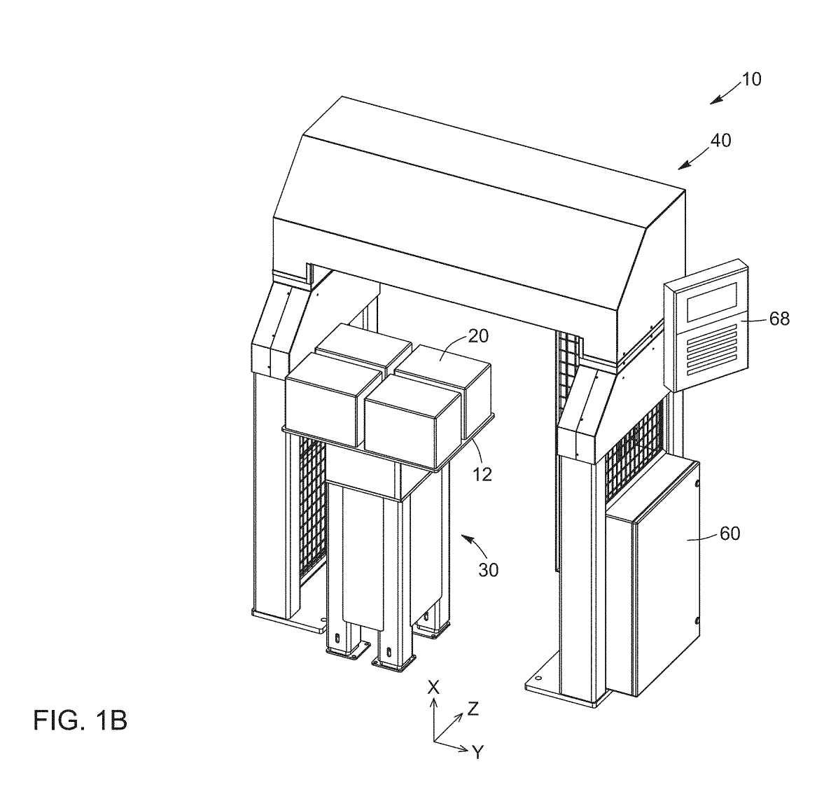

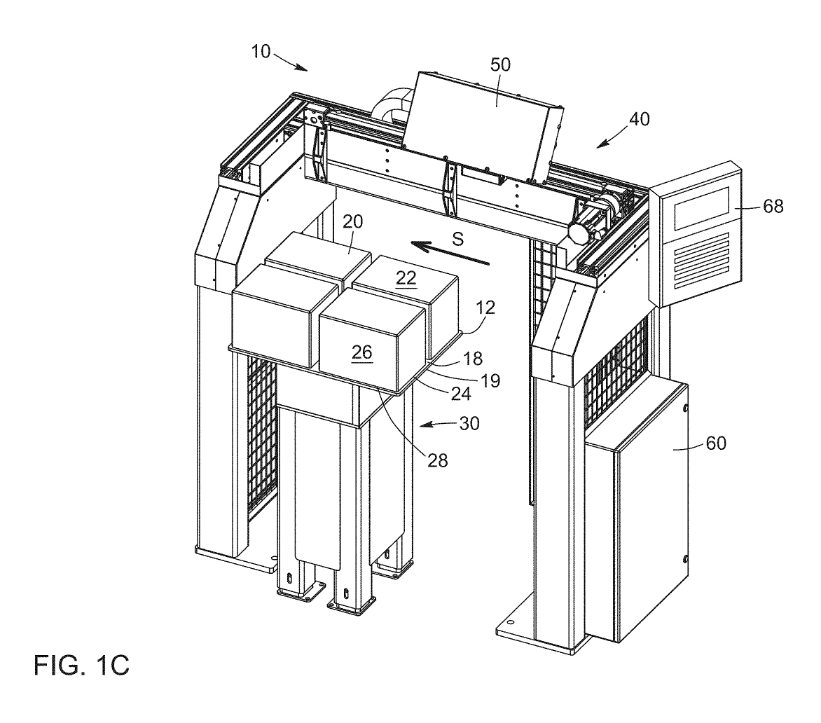

Referring to FIGS. 1A to 1C, there is shown a system 10 for performing the inspection of concrete blocks 20, in accordance with an embodiment. In the embodiment shown, the system 10 includes a weighing module 30 and a scanning module 40 cooperating to perform the inspection of concrete blocks 20, such that non-conform blocks, i.e. blocks 20 which aesthetic, dimensional and/or structural properties that depart from predetermined specifications thereof, can be identified. In other words, in an embodiment, the system 10 for performing the inspection of concrete blocks 20 measures parameters of the blocks 20 for variation in size, profile and/or density from the reference values of a specific block, to allow identification of non-conformity in the manufactured concrete blocks 20 and/or calibration of the components of a block manufacturing station manufacturing the concrete blocks 20.

In an embodiment, the weighing module 30 and the scanning module 40 are in data communication with a computing unit 60, for example and without being limitative, through physical connection between one another such as via a wire connection or the like. One skilled in the art will understand that, in an alternative embodiment, the components could also be connected by data transmission means other than physical connection, for example over a wireless network such as a personal area network (WPAN), a wireless local area network (WLAN), a wireless personal area network (PAN), or the like. As will be described in more details below, the computing unit 60 is configured to receive and store data from the weighing module 30 and the scanning module 40, process the data and generate and store inspection data relative to the concrete blocks 20 therefrom.

In the course of the present document, the term “concrete block” is used to refer to any manufactured building block made of concrete, such as concrete blocks or concrete pavers used to build walls or floors (e.g. patio floors, driveways or the like). It will be understood that, during inspection, the concrete blocks 20 can be in an uncured state or a cured state. In other words, in an embodiment, the system 10 is configured to perform inspection of uncured concrete blocks 20, but one skilled in the art will understand that, in an alternative embodiment, cured concrete blocks could also be inspected using the system described herein. The concrete block has an upper surface 22, a lower surface 28, side walls 26 extending between the upper surface 22 and the lower surface 28 and lower edges 24 defined at a junction of the side walls 26 and an upper surface 18 of a support plate 12 onto which the concrete block 20 is supported, as will be described below. The concrete block 20 can have a substantially plane upper surface 22 or a textured upper surface 22.

In an embodiment, the concrete blocks 20 are manufactured at a block manufacturing station (not shown), for example by filling a hollow mold (not shown) with a concrete mixture and compressing the concrete mixture using a compressor (not shown) to produce uncured concrete blocks 20. The manufactured uncured concrete blocks 20 are supported on a support plate 12 having an upper surface 18 and a lower surface 19. In an embodiment, more than one concrete block 20 are supported on a single support plate 12. For example and without being limitative, in the embodiment shown, four concrete blocks 20 are supported on the support plate 12. One skilled in the art will however understand that, in an alternative embodiment, a single concrete block 20 or more or less than four concrete blocks 20 can be supported on the support plate 12. For ease of description, reference to multiple concrete blocks 20 supported on the support plate 12 will be made in the description below.

The support plate 12 (with the concrete blocks 20 thereon) is conveyed in a conveying direction, on a conveyor 14 extending along a conveying axis C, towards the weighing module 30 and the scanning module 40. In the embodiment shown, the conveyor 14 includes conveyor belts 15 spaced apart from one another in a direction transverse to the conveying axis C and defining a free space 16 therebetween. As will be described in more details below, the free space 16 between the conveyor belts 15 allows engagement of the support plate 12 by the weighing module 30, from below, without contact between the weighing module 30 and the conveyor 14, thereby substantially isolating the weighing module 30 from the possible vibrations of the conveyor 14 which is often prone to vibration induced by the compactor compacting the blocks during manufacture thereof. One skilled in the art will understand that in an alternative embodiment (not shown) a different conveyor type or conveyor configuration allowing the weighing module 30 to engage the corresponding support plate 12, without contact with the conveyor 14 can be used. For example and without being limitative, in an alternative embodiment (not shown), the weighing module 30 can be positioned between two conveyor sections spaced apart from one another along the conveying axis C or could extend laterally beyond each side of the conveyor.

As mentioned above, the weighing module 30 is configured to engage a corresponding support plate 12, as the support plate 12 is conveyed thereabove by the conveyor 14, to weigh the support plate 12 and the concrete blocks 20 supported thereon. In other words, the weighing module 30 engages each one of the support plate 12 being conveyed thereabove by the conveyor and generates weigh data relative to the corresponding support plate 12.

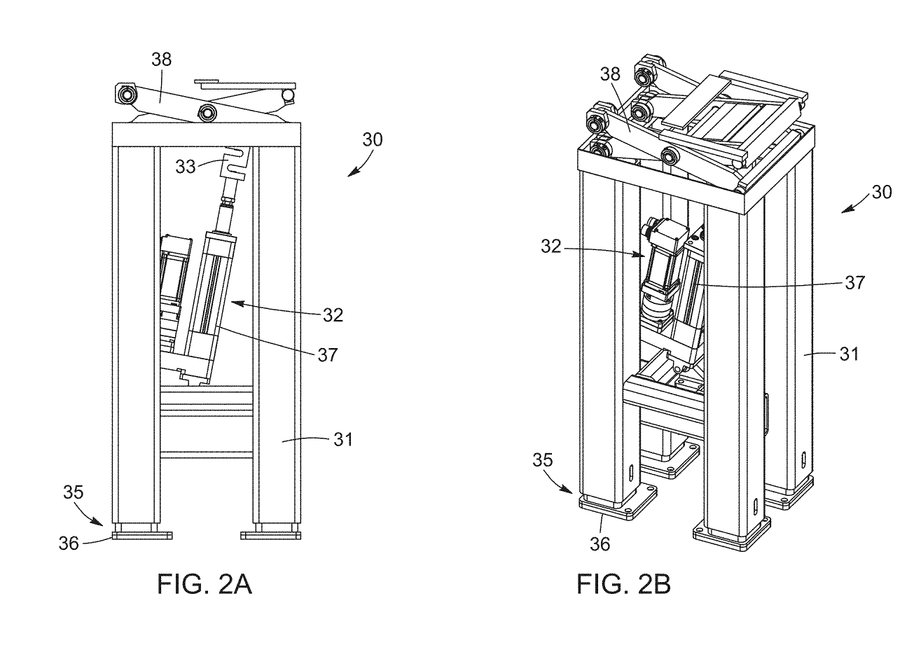

Referring to FIGS. 1A to 2B, in the embodiment shown, the weighing module 30 includes a body 31, a lifting mechanism 32 mounted to the body 31 and a load cell 33 mounted to the lifting mechanism 32.

In an embodiment, the lifting mechanism 32 includes a linear actuator 37 operatively connected to a scissor type elevator 38 for actuation thereof. The lifting mechanism 32 is movable between an inactive configuration and an active configuration. In the embodiment shown, in the inactive configuration, the linear actuator 37 is retracted and the scissor type elevator 38 is disengaged from the support plate 12. The lifting mechanism 32 is maintained in the inactive configuration until a support plate 12 is located in the proper position over the weighing module 30 (i.e. until the support plate 12 is substantially centered over the weighing module 30). In the embodiment shown, in the active configuration, the actuator 37 is extended and the scissor type elevator 38 is actuated upwardly to engage the support plate 12, from below, thereby lifting the support plate 12 away from the conveyor 14. In other words, in the operative configuration, the scissor type elevator 38 lifts the support plate 12 upwardly such that the support plate 12 is supported entirely by the weighing module 30 during a weighing time period where the support plate 12 and the concrete blocks supported thereon are weighed. Following the weighing time period, the lifting mechanism 32 is brought back in the inactive configuration, such that the support plate 12 is lowered back onto the conveyor 14 and can subsequently continue being conveyed in the conveying direction along the conveying axis C.

The load cell 33 is used to measure the force applied on the weighing module 30 by the support plate 12, when the lifting mechanism 32 is configured in the active configuration and the support plate 12 is supported entirely by the weighing module 30. The load cell 33 generates an electric signal as being converted in weight data indicative of the measure the weight of the support plate 12 and the concrete blocks 20 supported thereon. In the embodiment shown, the load cell 33 is a strain gauge load cell, but one skilled in the art will understand that, in alternative embodiments (not shown), other types of load cells, such as a hydraulic load cell, a pneumatic load cell, or the like can be used. One skilled in the art will also understand that other devices for weighing the support plate and the concrete blocks 20 and generating the weight data, when the entire weight thereof is applied on the weighing module 30 can be used.

In an embodiment, the weighing module 30 includes a vibration isolation assembly 35 operative to prevent or mitigate the propagation of vibrations from the environment (e.g. vibrations of the floor onto which the weighing module 30 is supported) to the support plate 12 engaged by the weighing module 30 (i.e. to prevent the transfer of vibration to the support plate 12) during the weighing time period. It will be understood that, since the support plate 12 is lifted away from the conveyor 14 during the weighing time period, the support plate 12 is also isolated from the vibrations of the conveyor 14 during the weighing time period.

In an embodiment, the vibration isolation assembly 35 includes resilient members 36 (or passive isolators), extending between the body 31 and the floor onto which the body 31 is supported to substantially absorb vibrations from the floor onto which the body 31 is supported. In the embodiment shown, the resilient members 36 are rubber pads, but one skilled in the art will understand that, in an alternative embodiment (not shown), the resilient members 36 could be a different component such as a mechanical spring or the like. Once again, one skilled in the art will understand that, in an alternative embodiment (not shown) the vibration isolation assembly 35 could be positioned differently than in the embodiment shown and/or be embodied by a different assembly, while still providing the desired vibration isolation of the support plate 12 engaged by the weighing module 30. For example and without being limitative, in an alternative embodiment (not shown), the vibration isolation assembly 35 could be an active vibration isolation assembly.

In an embodiment (not shown), the system 10 can include an additional weighing module positioned prior to the block manufacturing station (not shown) and configured to measure the weight of the empty support plates 12 (i.e. to measure the weight of a support plate 12 before the blocks 20 are loaded thereon). In an embodiment, the additional weighing module can be substantially similar to the above described weighing module 30, and therefore does not need to be described herein. One skilled in the art will understand that the additional weighing module could also be different from the above described weighing module 30 (i.e. it could be implemented using different mechanical components than the weighing module described above).

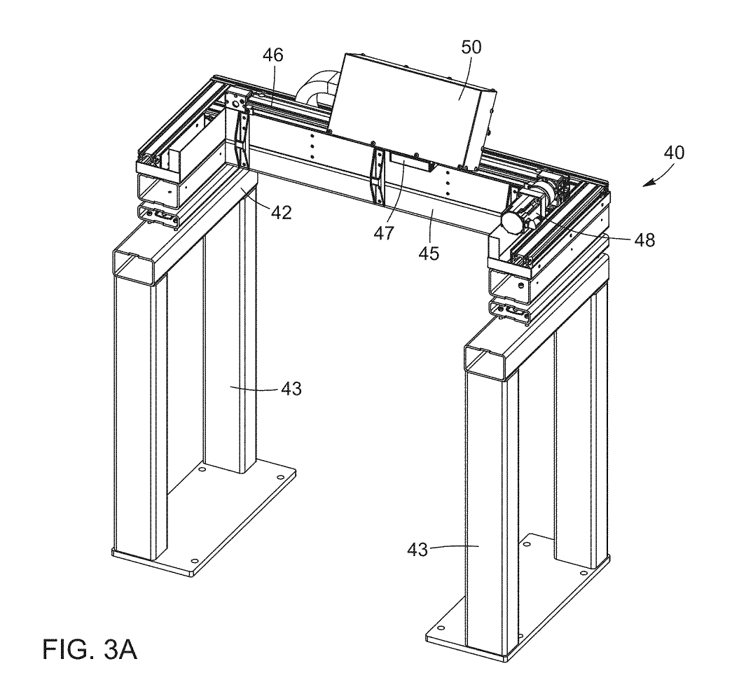

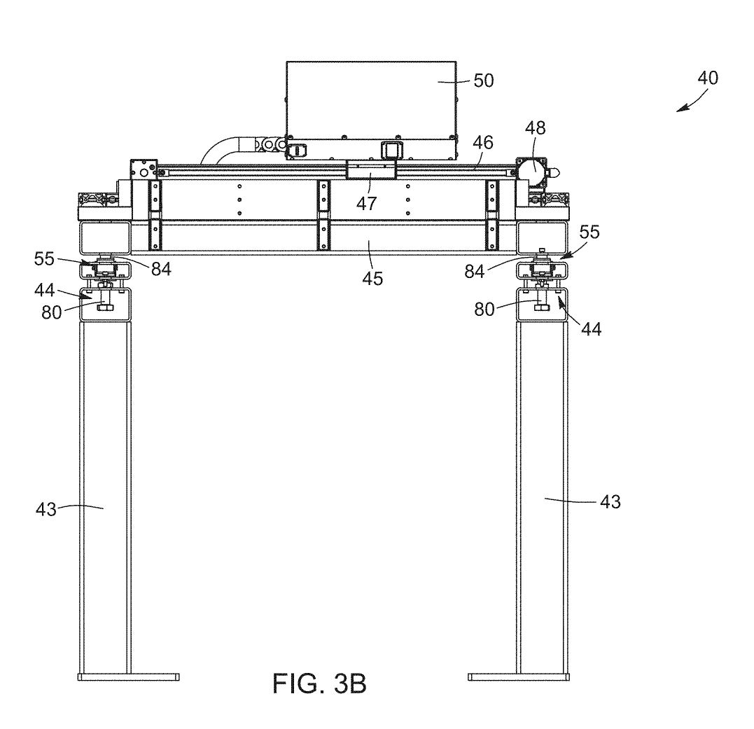

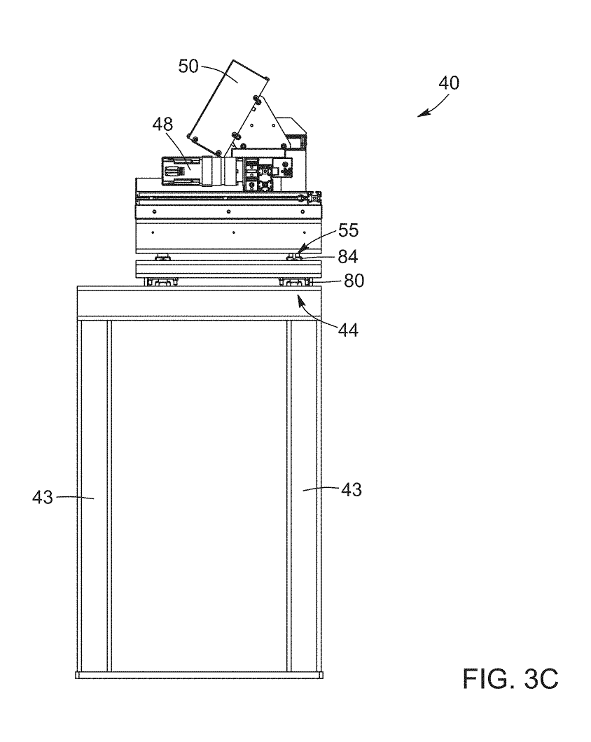

Referring to FIGS. 1A to 1C and 3A to 3C, as mentioned above, the system 10 for performing the inspection of concrete blocks 20 further includes a scanning module 40 configured to scan the inspected concrete blocks 20 while the concrete block remain static (i.e. while the concrete blocks 20 are immobile). The scanning module 40 is configured to scan the concrete blocks 20 supported on a support plate 12, from above, with an angled orientation with regard to a vertical axis X, as the concrete blocks 20 and the support plate 12 are positioned under the scanner 50 thereof and are immobile. As will be described in more details below, the scanning module 40 hence determines the shape of the upper surface 22 of each one of the concrete blocks and the shape of a corresponding peripheral section of the upper surface 18 of the upper plate 12 and generates surface scan data relative to each block 20 and the corresponding peripheral section of the support plate 12.

The scanning module 40 includes a support base 42 with a scanner 50 mounted thereto and supported thereon, over the conveyor 14, to provide a downward angled view of the support plate 12 and the concrete blocks 20 conveyed on the conveyor 14.

In the embodiment shown, the base 42 is an arch shaped structure including supporting legs 43 extending substantially vertically on opposed sides of the conveyor 14 and a transversal support 45 extending substantially transversally to the conveying axis C and connected to the supporting legs 43 at an upper end thereof. One skilled in the art will understand that, in an embodiment, any one of the supporting legs 43 and the transversal support 45 could include a plurality of components securable to one another to define the corresponding one of the supporting legs 43 and the transversal support 45. Conversely, in an alternative embodiment, any one of the supporting legs 43 and the transversal support 45, or the combination thereof, could be embodied in a single piece component (i.e. could be integral).

In an embodiment, the scanning module 40 includes a height adjustment mechanism 44 allowing a precise height adjustment and levelling of components of the base 42 and the scanner 50 connected thereto. In the embodiment shown, the height adjustment mechanism 44 includes threaded connectors 80 vertically connecting two vertically adjacent components of the base 42 and allowing the distance therebetween to be varied through screwing/unscrewing of the threaded connectors 80. One skilled in the art will however understand that, in an alternative embodiment (not shown), the height adjustment mechanism 44 could be positioned differently than in the embodiment shown. Moreover, several types and configurations of height adjustment mechanisms 44 are known in the art and, consequently, in an alternative embodiment (not shown), could be used to provide the desired precise height adjustment and levelling of components of the base 42.

In an embodiment, the scanning module 40 also includes a vibration isolation assembly 55 configured to absorb vibrations from the environment, therefore substantially preventing or mitigating the transfer of the vibrations (e.g. vibrations from the floor onto which the support legs 43 are rested on) to the scanner 50. As will be understood by those skilled in the art, the vibration isolation assembly 55 thereby substantially prevent the vibrations of the ambient environment from affecting the measures taken by the scanner 50. In the embodiment shown, the vibration isolation assembly 55 includes resilient members 84 (or passive isolators), extending between two vertically adjacent components of the base 42 to substantially absorb vibrations from the floor onto which the support legs 43 are supported. In the embodiment shown, the resilient members 84 are rubber pads, but one skilled in the art will understand that, in an alternative embodiment (not shown), the resilient members could be a different component such as a mechanical spring or the like. Once again, one skilled in the art will understand that, in an alternative embodiment (not shown) the vibration isolation assembly 55 could be positioned differently than in the embodiment shown and/or be embodied by a different assembly, while still providing the desired vibration isolation of the scanner 50. For example and without being limitative, in an alternative embodiment (not shown), the vibration isolation assembly 55 could be an active vibration isolation assembly.

In the embodiment shown, the scanner 50 is displaceably mounted to the base 42 and is displaceable with regard to the base 42 along a scanning axis S. In the embodiment shown, the scanning axis S extends substantially transversely to the conveying axis C, but one skilled in the art will understand that, in an alternative embodiment (not shown) the scanning axis S could extend in a different direction. In the embodiment shown, the base 42 includes a guide rail 46 extending along the scanning axis S. In an embodiment, the guide rail 46 is secured to the transversal support 45 of the base 42. However, one skilled in the art will understand that, in an alternative embodiment, the guide rail 46 can be integral to the transversal support 45.

To allow displacement of the scanner 50 with regard to the base 42, in the embodiment shown, the scanner 50 is secured to a slider 47 slidably engaged with the guide rail 46. In an embodiment, the scanning module 40 further includes a linear actuator 48 engaged between the scanner 50 and the base 42 and configured to move the scanner 50 along the scanning axis S. In the embodiment shown, the linear actuator 48 is an electro-mechanical actuator, but one skilled in the art will understand that, in alternative embodiments (not shown), the actuator 48 can be another type of linear actuator, such as a piezoelectric actuator or the like.

In the embodiment shown, the scanner 50 is a triangulation based 3D laser scanner including a combination of a laser source (not shown) producing a laser beam 52 towards the concrete blocks 20 and a camera (not shown) sensing the laser beam. One skilled in the art will however understand that, in an alternative embodiment, other laser technology allowing a 3D scan of a surface of the concrete blocks 20 and a section of the upper surface of the support plate 12, such as a time of flight laser device or the like, could also be used. Moreover, in other alternative embodiments (not shown), the scanner could be a scanner using a scanning technology different from a laser scanning technology such as stereo scanner, a stereo active scanner, an interferometry scanner or the like.

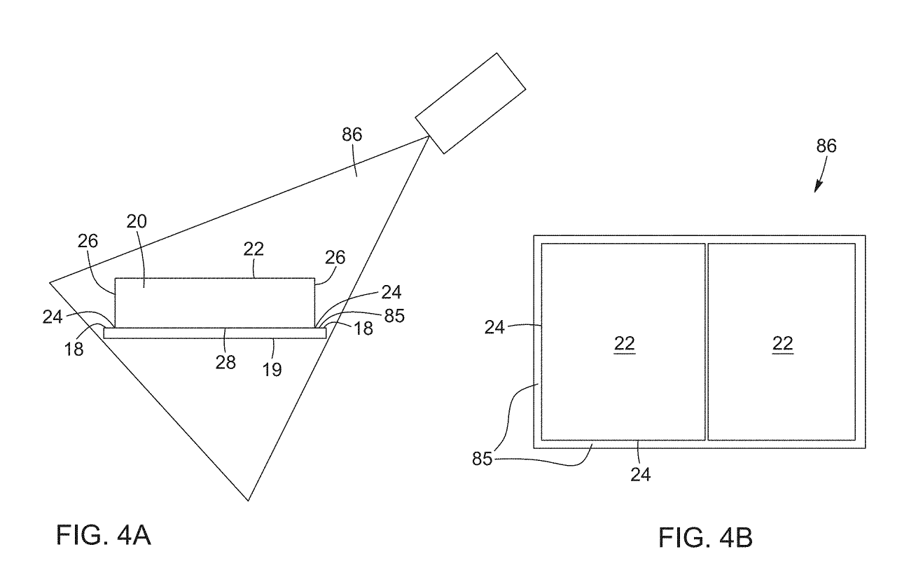

With reference to FIGS. 4A and 4B, the scanner 50 is positioned and oriented to have a scanning range 86 covering the entire upper surface 22 of each concrete block 20 supported on the support plate 12 and a section 85 of the upper surface 18 of the support plate 12 proximal to at least two consecutive lower edges 24 of at least one of the concrete block 20 supported on the support plate 12. Each lower edge 24 of a concrete block 20 is located at a junction of the upper surface 18 of the support plate 12 and the corresponding side wall 26 of the concrete block 20. Hence, in other words, the scanning range 86 of the scanner 50 encompasses the entire upper surface 22 of each concrete block 20 and a section 85 of the upper surface 18 of the support plate 12 extending from two consecutive lower ends of side walls 26 of at least one of the concrete blocks 20 (i.e. a section of the upper surface 18 of the support plate 12 extending from two consecutive junctions of side wall 26 of the concrete block with the upper surface 18 of the support plate 12, for at least one concrete block 20).

One skilled in the art will understand that, in the embodiment shown, the scanning range 86 of the scanner 50 is defined by the combination of the instant field of views (or slices) of the scanner 50 relative to the laser beam 52 at each one of the linear positions of the scanner 50, as it is displaced along the scanning axis S. In other words, the scanning range 86 of the scanner 50 is defined by the addition of all the instant field of views of the scanner 50 during the linear displacement thereof. To result in the above described scanning range, the scanner 50 is required to have an angled configuration regarding a vertical axis X and thereby producing instant field of views limiting occlusion, i.e. the angled configuration should result in instant field of views of the scanner 50 together covering the upper surface 22 of each concrete block 20 supported on the support plate 12 and the section of the upper surface 18 of the support plate 12 extending from the at least two consecutive lower edges 24 of the at least one of the concrete block 20 supported on the support plate 12 during the linear displacement of the scanner.

In view of the above, upon displacement along the scanning axis S, the scanner 50 scans the upper surface 22 of the concrete blocks 20 and the section of the upper surface 18 of the support plate 12 proximal to at least two consecutive lower edges 24 of the concrete blocks 20 and generates surface scan data for each concrete block 20 supported on the support plate 12. The generated surface scan data includes the XYZ coordinates of each surface point defining the upper surface 22 of the concrete blocks 20 supported on the support plate 12 and the XYZ coordinates of each surface point defining the section of the upper surface 18 of the support plate 12 proximal to at least two consecutive lower edges 24 of at least one of the concrete block 20 supported on the support plate 12.

In the embodiment shown, the scanning module 40 and the weighing module 30 are substantially aligned along the conveying axis C, thereby allowing the weighing and the scanning of each concrete blocks 20 supported on a support plate 12 to be performed simultaneously. In other words, the scanning module 40 and the weighing module 30 are positioned such that the scanning module 40 scans the concrete blocks 20 supported on a support plate 12 during the weighing time period thereof (i.e. while the support plate 12 is engaged from below by the weighing module 30 and is lifted away from the conveyor 14 such that the support plate 12 is supported entirely by the weighing module 30).

The simultaneous weighing of the support plate 12 (and concrete blocks supported thereon) by the weighing module 30 and scan of the concrete blocks 20 supported on the support plate 12 by the scanning module 40 has several advantages. For example and without being limitative, it limits the time period where the support plate 12 is maintained static along the conveying axis C to perform the inspection, thereby limiting the impact of the inspection process on the production time of the concrete blocks 20. Moreover, the scanning of the at least one concrete block 20 supported on the support plate 12, while the support plate 12 is lifted away from the conveyor 14, such that the support plate 12 is supported entirely by the weighing module 30, allows the scanning to be performed with minimal interference due to vibration, given that when the support plate 12 is lifted away from the conveyor 14 it is substantially isolated from the vibrations, as described in more details above.

As mentioned above, in an embodiment, the system 10 further includes a computing unit 60 in data communication with the weighing module 30 and the scanning module 40. The computing unit 60 receives and stores the weight data from the weighing module 30 and the surface scan data from the scanning module 40, processes the data according to a set of instructions and generates and stores inspection data relative to the concrete blocks 20 therefrom. In an embodiment, the processing unit 60 includes a processor, a memory, a data storage device, communication hardware and software and any other required components and/or circuitry for receiving and storing data, processing the received data and generating and storing additional data therefrom. In an embodiment, the computing unit 60 includes input devices and output devices. For example and without being limitative, in the embodiment shown, the computing unit 60 includes a display monitor 68 for displaying the inspection data, such as a topographic view (or heightmap) of the upper surface of the concrete blocks 20, to the user. In the embodiment shown, the computing unit 60 is embodied by a single computer, but one skilled in the art will understand that, in an alternative embodiment (not shown), the computing unit 60 can include a computer system including multiple interconnected computers in a network, where the resources can be distributed over the computer system.

In an embodiment, as described above, the weight data from the weighing module 30 includes the total weight values representative of the sum of the weight of the support plate 12 and the weight of the concrete blocks 20 supported thereon. Hence, the total weight values of the weight data need to be processed to determine the weight of the concrete blocks 20.

In an embodiment, to determine the weight of the concrete blocks 20, the support plate 12 is attributed a predetermined fixed weight, for example and without being limitative a weight supplied by the manufacturer or an average weight of an empty support plate 12. The predetermined fixed weight can be stored in the memory and/or data storage device of the computing device 60 or in a remote data storage device accessible by the computing device 60. Hence, the predetermined weight of the support plate can be subtracted from the total weight value representative of a measured weight of a corresponding support plate 12 (with concrete blocks 20 thereon) measured by the weighing module 30, to generate a block weight (i.e. weight data relative to only the concrete blocks 20 supported on the support plate 12).

In an alternative embodiment, the weight of the support plate 12 can be considered substantially marginal with regards to the weight of the concrete blocks 20 supported thereon, such that the weight of the support plate 12 is not taken into account in the processing of the total weight values (i.e. the weight of the support plate 12 is simply included in the block weight).

In an another alternative embodiment, where, as described above, the system 10 includes an additional weighing module (not shown) positioned prior to the block manufacturing station (not shown) and configured to measure the weight of the empty support plates 12, the measured weight of each specific empty support plate 12 can be transmitted to the computing unit 60 (i.e. the additional weighing module can be in data communication with the computing unit 60 and the weight of the empty support plates 12 can be part of the weight data). The weight of the empty support plates 12 can be stored in the memory and/or data storage device of the computing device 60 or in a remote data storage device accessible by the computing device 60. In such an embodiment, the measured weight of the specific empty support plate can be subtracted from the total weight value representative of the measured weight of a corresponding support plate 12 (with the blocks 20 thereon) measured by the weighing module 30, to generate a block weight having a greater precision.

In an embodiment where a plurality of concrete blocks 20 are supported on the support plate 12, it will be easily understood that the block weight can be processed by dividing the remaining weight by the number of blocks 20 supported on the support plate 12 to determine the weight of each one of the blocks 20. It will be understood that, to allow such extrapolation of the weight of each one of the plurality of blocks 20 supported on a single support plate 12, the plurality of blocks 20 are required to be substantially similar to one another (i.e. have substantially a same weight).

In an embodiment, the surface scan data is processed to determine if the XYZ coordinates of each surface point defining the upper surface 22 of each concrete block 20 is conform to the bloc specifications. In an embodiment, during processing, the XYZ coordinates of each surface point defining the upper surface 22 of each concrete block 20 is compared to a corresponding reference XYZ coordinate of a surface point defining the upper surface 22 of the specific concrete block 20, to generate inspection data relative to the upper surface 22 of the specific concrete block 20. In such an embodiment, the inspection data relative to the upper surface 22 of the specific concrete block 20 can indicate for each surface point defining the upper surface 22 of each concrete block 20 or for a group of surface points defining a section of the upper surface 22 if the surface is conform to the specification or non-conform therewith.

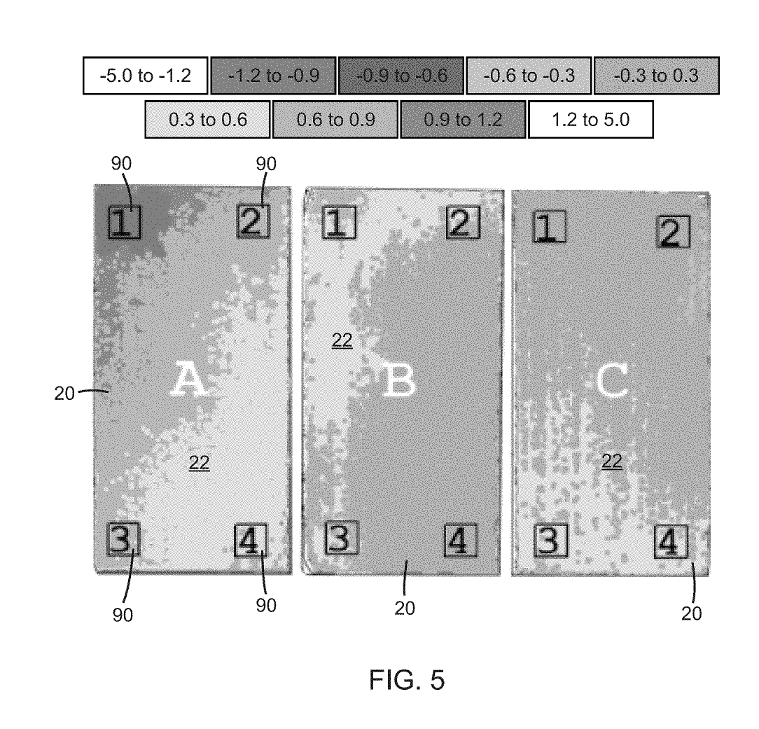

With reference to FIG. 5, for example and without being limitative, in an embodiment, the inspection data relative to the upper surface 22 of the specific concrete block 20 can be used to define a topographic view (or heightmap) of the upper surface 22 of the concrete block 20, with a color code indicative of whether the surface is substantially evenly leveled with the specification, higher than a predefined threshold with regard to the specification or lower than a predefined threshold with regard to the specification. The topographic view of the upper surface 22 of the concrete block 20 can be displayed on the display screen 68 of the computing unit 60 to provide feedback to the user. For example, sections of the upper surface 22 of the specific concrete block 20 can be colored in green if substantially evenly leveled with the specification, colored in varying shades of yellow, orange and red if higher than successive predefined thresholds with regard to the specification (e.g. yellow if higher than the specification of between 0.3 and 0.6 mm; orange if higher than the specification of between 0.6 and 0.9 mm; light red if higher than the specification of between 0.9 and 1.2 mm; and dark red if higher than the specification of between 1.2 and 5.0 mm) and colored in varying shades of blue, purple and white if lower than successive predefined thresholds with regard to the specification (e.g. blue if lower than the specification of between 0.3 and 0.6 mm; light purple if lower than the specification of between 0.6 and 0.9 mm; dark purple if lower than the specification of between 0.9 and 1.2 mm; and white if lower than the specification of between 1.2 and 5.0 mm). One skilled in the art will understand that in alternative embodiments(s) different color codes can be used and/or additional colors corresponding to additional thresholds can be used.

In an embodiment, the reference XYZ coordinate for each one of the specific surface points defining the upper surface 22 of the specific concrete block 20 are taken from a CAD model of the concrete bloc. In an alternative embodiment, the reference XYZ coordinates for each one of the specific surface points defining the upper surface 22 of the specific concrete block 20 can be derived from a prior scan of a concrete block 20 known to be conform to the specifications and which can be referred to as a “teach block”. In both cases the data from the CAD model or the scan of the teach block can be stored in the memory and/or data storage device of the computing device 60 or in a remote data storage device accessible by the computing device 60 as reference data (reference XYZ coordinate for each one of the specific surface points defining the upper surface 22 of a specific concrete block 20).

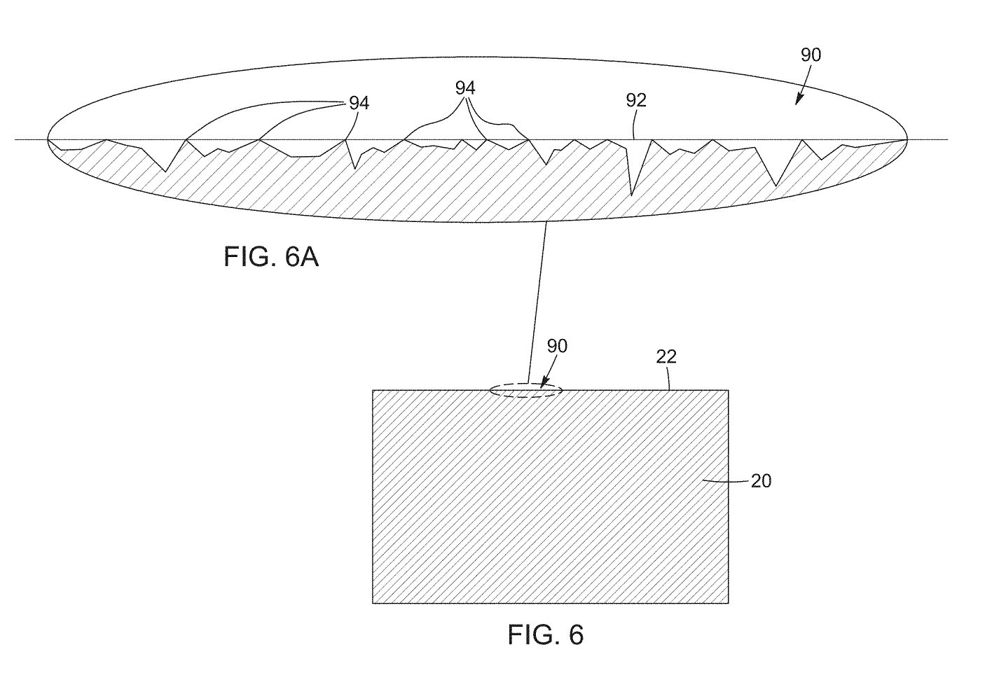

Referring to FIGS. 5, 6 and 6A, in an embodiment, the computing unit 60 is configured to further process the surface scan data corresponding to the XYZ coordinates of surface points defining predetermined measure section(s) 90 of the upper surface 22 of each concrete block 20 and generate a topmost surface 92 relative to the predetermined measure section(s) of the upper surface 22. The topmost surface 92 relative to the predetermined measure section(s) of the upper surface 22 correspond to a virtually defined substantially planar surface aligned on top of a set of topmost surface points 94 (i.e. topmost XYZ coordinates or summits of the surface points of the upper surface 22) for each predetermined measure section 90 and generally defining the upper surface thereof. In other words, the topmost surface 92 corresponds to the measure that would result from a manual measure using a mechanical instrument, not taking into account the porosity of the concrete. Indeed, the topmost surface 92 is not impacted by the porosity of the concrete in the predetermined measure section(s) as it relates only to the surface defined by the topmost surface points 94 in the predetermined measure section(s) 90. One skilled in the art will understand that, in an embodiment (not shown) a filtering of irregular topmost surface points of the predetermined measure section(s) can be performed to filter any topmost surface point(s) deviating from a substantially even set of topmost surface point(s), thereby preventing only a few deviant topmost surface points from negatively impacting the quality of the generated topmost surface.

In an embodiment, the computing unit 60 compares the generated topmost surface 92 of each predetermined measure section 90 of the concrete block 20 to a corresponding reference topmost surface of the specific concrete block 20, to generate further inspection data relative to the upper surface 22 of the specific concrete block 20. In such an embodiment, the inspection data relative to the upper surface 22 of the specific concrete block 20 can indicate, for each predetermined measure section 90, if the surface is conform to the specification or non-conform therewith. Once again, in an embodiment, the reference topmost surface can be taken from a CAD model of the concrete block 20 or a prior scan of the teach block and can be stored in the memory and/or data storage device of the computing device 60 or in a remote data storage device accessible by the computing device 60 as reference data.

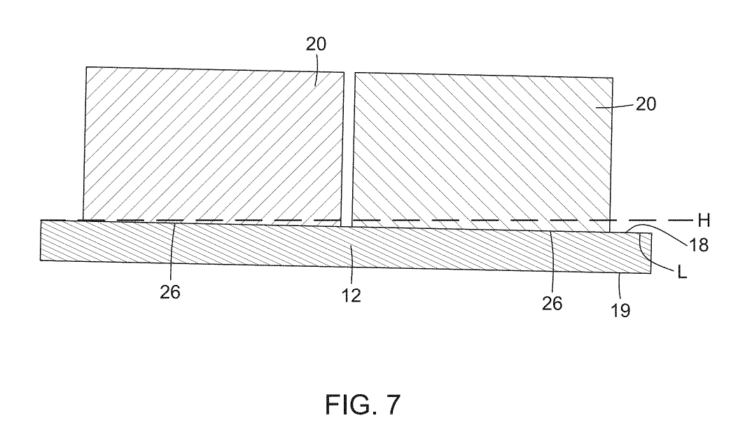

Referring to FIG. 7, in an embodiment, the computing unit 60 is configured to process the surface scan data corresponding to the XYZ coordinates of each surface point defining the section of the upper surface 18 of the support plate 12 proximal to the at least two consecutive lower edges 24 of the concrete block 20 and generate or define the lower block surface plane L therefrom. To generate the lower block surface plane L, the computing unit 60 determines the plane defined by the upper surface 18 of the support plate 12 in the sections proximal to the at least two consecutive lower edges 24 of the concrete block 20 and extends this plane to the section of the support plate 12 positioned under the concrete block 20. It will be understood that the upper surface 18 of the support plate 12 positioned under the concrete block 20 corresponds to the lower surface 28 of the concrete block 20. In an embodiment, once the lower block surface plane L has been generated, the computing unit 60 can generate XYZ coordinates for each surface point defining the lower surface of the concrete block 20. It will be understood that, by generating the lower block surface plane L using the XYZ coordinates of each surface point defining the section of the upper surface 18 of the support plate 12 proximal to at least two consecutive lower edges 24 of the concrete block 20, the volume of the concrete block 20 can be defined with a greater precision, given that otherwise the plane is estimated, such as being assumed to be a substantially horizontal plane H, which can differ from the real lower block surface plane L.

In order to determine the volume of the concrete block 20, the computing unit can subsequently determine the height difference between each surface point defining the upper surface 22 of the concrete block 20 and the corresponding surface point defining the lower surface of the concrete block 20, the block volume being defined by the addition of the measured height difference between each corresponding lower and upper surface points.

Using the now known block volume and block weight, the computing unit 60 can generate inspection date relative to the density of the concrete block 20. In an embodiment, the calculated density can be compared to a reference density value, to further determine if the concrete block 20 is conform to the specification. Once again, in an embodiment, the inspection data relative to the density of the specific concrete block 20 can indicate if the block 20 is conform to the specification or non-conform therewith. In an embodiment, the reference density data can be a predetermined reference data or data calculated through a prior scan and weighing of the teach block, with the data being stored in the memory and/or data storage device of the computing device 60 or in a remote data storage device accessible by the computing device 60.

It will be understood that the inspection data can be displayed on the display screen 68 of the computing unit 60 to provide user feedback regarding each one of the manufactured concrete block 20. The inspection data can also be stored as data to be subsequently used regarding the quality of the manufactured block and/or functioning or calibration of the components of the devices used for manufacturing the blocks 20. In an embodiment, the computing unit 60 can also be in data communication with devices used for manufacturing the blocks 20, the inspection data being used to calibrate or fine tune the devices used for manufacturing the blocks 20 to ensure constant quality and conformity of the manufactured concrete blocks 20.

In an embodiment, the inspection of the uncured concrete blocks 20, transmission of the weighing data from the weighing module 30 and the surface scan data from the scanning module 40 to the computing unit 60, generation of the inspection data by the computing unit and display of the inspection data on the display screen 68 of the computing unit 60 are performed in real-time or near real-time, i.e. they are performed substantially instantly as each one of the blocks 20 are conveyed through the weighing module 30 and/or scanning module 40.

In an embodiment, the computing unit 60 operates as a controller to coordinate the operation of the weighing module 30 (e.g. the operation of the lifting mechanism thereof), the scanning module 40 (e.g. the operation of the scanner and the linear actuator 48 thereof) and the other components of the system 10, such as the conveyor 14, the block manufacture module (not shown), or the like. One skilled in the art will understand that, in alternative embodiments, additional controllers could be used for coordinating some of the components of the system 10.

The system 10 for performing the inspection of concrete blocks 20 having been described in details above, a method to perform inspection of concrete blocks 20 using the above described system will now be described in more details below.

In an embodiment, the method includes the steps of conveying a support plate 12 and concrete blocks 20 supported thereon along a conveying axis C; weighing the support plate 12 with the concrete blocks 20 supported thereon to generate weight data; scanning an upper surface 22 of the concrete blocks 20 and the upper surface 18 of the support plate 12 as the concrete blocks 20 and the support plate 12 are maintained static to generate surface scan data; and processing the weight data and the surface scan data according to a set of instructions to generate inspection data indicative of a conformity or non-conformity of each one of the concrete blocks 20 with specifications thereof.

As mentioned, the support plate 12 has an upper surface 18 and the concrete blocks 20 each have an upper surface 22, a lower surface 28, side walls 26 extending between the upper surface 22 and the lower surface 28 and lower edges 24 defined at a junction of the side walls 26 and the upper surface 18 of the support plate 12. The scanning covers an entirety of the upper surface 22 of the concrete blocks 20 supported on the support plate 12 and a section of the upper surface 18 of the support plate 12 extending from at least two consecutive ones of the lower edges 24 of at least one concrete block 20 supported on the support plate 12 and generate surface scan data including the XYZ coordinates of each surface point defining the upper surface 22 of the concrete blocks 20 supported on the support plate 12 and the XYZ coordinates of each surface point defining the section of the upper surface 18 of the support plate 12 extending from at least two consecutive ones of the lower edges 24 of at least one concrete block 20 supported on the support plate 12.

In an embodiment, as described above, the support plate 12 is engaged from below and is lifted away from the conveyor 14 onto which it conveyed during a weighing time period to perform the weighing of the support plate 12 and the concrete blocks 20 supported thereon. In an embodiment, the support plate 12 is substantially isolated from vibrations during the weighing time period to prevent the measure taken to be hindered by the vibrations from the environment.

In an embodiment, the weighing of the support plate 12 with the concrete blocks supported thereon and the scanning performed by the displacement of the scanner along the scanning axis to scan the upper surface of the concrete block and the upper surface of the support plate are performed simultaneously.

In an embodiment, the step of processing the weight data and the surface scan data according to a set of instructions includes defining the topmost surface points 94 of a predetermined measure section 90 and generating a topmost surface 92 (i.e. a virtually defined substantially planar topmost surface) aligned on top of the topmost surface points 94. Details of the steps performed to generate the topmost surface 92 are given above and need not be repeated herein.

In an embodiment, the step of processing the weight data and the surface scan data according to a set of instructions includes determining a volume and a weight of the concrete block 20, the volume and the weight of the concrete block being used to determine a density of the concrete block 20. In an embodiment, the determination of the volume of the concrete block 20 includes defining a plane L of the lower surface 28 of the concrete block 20 using the scan surface data relative to the section of the upper surface 18 of the support plate 12 extending from the at least two consecutive ones of the lower edges 24 of at least one concrete block 20 supported on the support plate 12; generating XYZ coordinates for surface points defining the lower surface 28 of the concrete block 20 extending along the defined plane of the lower surface of the concrete block 20; and calculating a sum of the height differences between each one of the surface points defining the upper surface 22 of the concrete block 20 and a corresponding one of the surface points defining the lower surface 28 of the concrete block 20 (i.e. calculating the sum of the height differences between each one of the surface points defining the upper surface 22 of the concrete block 20 and the corresponding surface points defining the lower surface 28 of the concrete block 20 and vertically aligned therewith).

Several alternative embodiments and examples have been described and illustrated herein. The embodiments of the invention described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while specific embodiments have been illustrated and described, numerous modifications come to mind without significantly departing from the scope of the invention as defined in the appended claims.

1. A system for performing the inspection of a concrete block, the system comprising:
a conveyor extending along a conveying axis and conveying a support plate having an upper surface and the concrete block supported thereon, the concrete block having an upper surface, a lower surface, side walls extending between the upper surface and the lower surface and lower edges defined at a junction of the side walls and the upper surface of the support plate;
a weighing module weighing the support plate with the concrete block supported thereon; and
a scanning module including a scanner scanning the upper surface of the concrete block and the upper surface of the support plate, the scanner being displaceable along a scanning axis and having a scanning range covering an entirety of the upper surface of the concrete block supported on the support plate and a section of the upper surface of the support plate extending from at least two consecutive ones of the lower edges of the concrete block supported on the support plate.

2. The system of claim 1, wherein the weighing module engages the support plate being conveyed on the conveyor from below and the scanner is positioned above the support plate being conveyed on the conveyor and the concrete block supported thereon.

3. The system of claim 2, wherein the weighing module includes a lifting mechanism configured to lift the support plate away from the conveyor during a weighing time period where the weighing module performs the weighing of the support plate with the concrete block supported thereon.

4. The system of claim 3, wherein the weighing module includes a vibration isolation assembly substantially isolating the support plate engaged by the weighing module from vibrations, during the weighing thereof.

5. The system of claim 4, wherein the weighing module and the scanning module are substantially aligned along the conveying axis to perform the scanning of the upper surface of the concrete block and the support plate during the weighing time period.

6. The system of claim 5, wherein the scanning module includes a support base with the scanner mounted thereto and supported thereon, the support base comprising a vibration isolation assembly substantially isolating the scanner from vibrations.

7. The system of claim 1, wherein the support base includes a guide rail extending along the scanning axis and the scanner is secured to a slider slidably engaged with the guide rail, the scanning module further comprising a linear actuator connected between the scanner and the base and configured to move the scanner along the scanning axis.

8. The system of claim 1, wherein the weighing module generates weight data and the scanning module generates surface scan data, the system further comprising a computing unit in data communication with the weighing module and the scanning module, the computing unit receiving the weight data from the weighing module and the surface scan data from the scanning module, processing the weight data and the surface scan data according to a set of instructions and generating inspection data indicative of a conformity or non-conformity of the concrete block.

9. The system of claim 8, wherein the concrete block supported on the support plate includes a predetermined measure section of the upper surface thereof, the computing unit being configured to process the surface scan data according to the set of instructions to define a set of topmost surface points of the predetermined measure section and to generate a virtually defined substantially planar topmost surface aligned on top of the set of topmost surface points.

10. The system of claim 8, wherein the computing unit is configured to process the weight data and the surface scan data according to the set of instructions to determine a volume and a weight of the concrete block, the volume and the weight of the concrete block being used to determine a density of the concrete block.

11. The system of claim 10, wherein, to determine the volume of the concrete block, the computing unit is configured to process the surface scan data according to the set of instructions to define a plane of the lower surface of the concrete block from the surface scan data relative to the section of the upper surface of the support plate extending from the at least two consecutive ones of the lower edges of the concrete block supported on the support plate, generate XYZ coordinates for surface points defining the lower surface of the concrete block extending along the defined plane thereof and calculate a sum of height differences between each surface point defining the upper surface of the concrete block and a corresponding one of the surface points defining the lower surface of the concrete block.

12. A method for performing the inspection of a concrete block, the method comprising:
conveying a support plate having an upper surface and a concrete block supported thereon along a conveying axis, the concrete block having an upper surface, a lower surface, side walls extending between the upper surface and the lower surface and lower edges defined at a junction of the side walls and the upper surface of the support plate;
weighing the support plate with the concrete block supported thereon; and
displacing a scanner along a scanning axis to scan the upper surface of the concrete block and the upper surface of the support plate as the concrete block and the support plate remain static, the scan covering an entirety of the upper surface of the concrete block supported on the support plate and a section of the upper surface of the support plate extending from at least two consecutive ones of the lower edges of the concrete block supported on the support plate.

13. The method of claim 12, wherein the step of weighing the support plate with the concrete block supported thereon includes engaging the support plate from below.

14. The method of claim 13, wherein the support plate is conveyed on a conveyor and wherein the step of weighing the support plate with the concrete block supported thereon includes lifting the support plate away from the conveyor during a weighing time period.

15. The method of claim 14, wherein the step of weighing the support plate with the concrete block supported thereon and the step of displacing a scanner along the scanning axis to scan the upper surface of the concrete block and the upper surface of the support plate are performed simultaneously.

16. The method of claim 15, wherein the step of weighing the support plate with the concrete block supported thereon includes substantially isolating the support plate from vibrations during the weighing time period.

17. The method of claim 12, wherein the step of weighing the support plate with the concrete block supported thereon further comprises generating weight data and the step of displacing the scanner along the scanning axis to scan the upper surface of the concrete block and the upper surface of the support plate further comprises generating scan surface data, the method further comprising processing the weight data and the surface scan data according to a set of instructions and generating inspection data indicative of a conformity or a non-conformity of the concrete block with specifications thereof.

18. The method of claim 17, wherein the concrete block supported on the support plate includes a predetermined measure section of the upper surface thereof and wherein the step of processing the weight data and the surface scan data according to a set of instructions comprises defining a set of topmost surface points of the predetermined measure section and generating a virtually defined substantially planar topmost surface aligned on top of the set of topmost surface points.

19. The method of claim 17, wherein the step of processing the weight data and the surface scan data according to a set of instructions comprises determining a volume and a weight of the concrete block, the volume and the weight of the concrete block being used to determine a density of the concrete block.

20. The method of claim 19, wherein the step of determining a volume of the concrete block includes determining a plane of the lower surface of the concrete block using the scan surface data relative to the section of the upper surface of the support plate extending from the at least two consecutive ones of the lower edges of the concrete block supported on the support plate, generating XYZ coordinates for surface points defining the lower surface of the concrete block extending along the plane thereof and calculating a sum of the height differences between each surface point defining the upper surface of the concrete block and a corresponding one of the surface points defining the lower surface of the concrete block.
